(19) United States
(12) Patent Application Publication
(10) Pub. No.: US 2026/0224569 A1
(43) Pub. Date: Aug. 6, 2026

(54) STABLE PARENTERAL FORMULATIONS OF FLUCYTOSINE

(71) Applicant: CIPLA LIMITED, Mumbai, Maharashtra (IN)

(72) Inventors: Pradeep BHADAURIA, Weston, FL (US); Arunprasath KALIAPERUMAL, Weston, FL (US); Tushar RAJYAGURU, Bhavnagar, Gujarat (IN); Harshad HARDE, Nashik, Maharashtra (IN)

(21) Appl. No.: 19/154,237

(22) PCT Filed: Feb. 7, 2024
(86) PCT No.: PCT/IN2024/050119

§ 371 Date: Aug. 6, 2025

(30) Foreign Application Priority Data
Feb. 14, 2023 (IN) 202321009752

(51) Int. Cl. A61K 31/513; A61J 1/06; A61K 9/08; A61K 47/02; A61K 47/18

(52) U.S. Cl. CPC A61K 31/513; A61K 9/08; A61K 47/02; A61K 47/18; A61J 1/065

(57) ABSTRACT

A stable, sterile, ready-to-use aqueous pharmaceutical formulation comprising flucytosine or its pharmaceutically acceptable salts and pharmaceutically acceptable excipients in glass container for intravenous and intraperitoneal administration. The formulation is manufactured by aseptic fill finish and filled in suitable presterilized glass container. The formulation is suitable for treating systemic yeast and fungal infections.

### FIELD OF INVENTION

The present invention relates to stable, sterile, ready-to-use aqueous pharmaceutical formulation comprising flucytosine or its pharmaceutically acceptable salts, and pharmaceutically acceptable excipients. The formulation is suitable for parenteral administration and is provided in a glass container. The invention further relates to the method of manufacture and sterilization of such formulations and using the formulation for treating systemic yeast and fungal infections.

### BACKGROUND OF INVENTION

Annually, over 150 million severe cases of fungal and yeast infections occur worldwide, resulting in approximately 17 million deaths per year. Alarmingly, these numbers are continuously on the rise with several social and medical developments during the past decades that have abetted the spread of fungal and yeast infections. These infections are increasingly becoming a global health problem that is associated with high morbidity and mortality rates as well as with devastating socioeconomic consequences. Such infections are primarily cryptococcosis, candidiasis, chromomycosisc etc. The yeasts Candida and Criptococcus spp. are important human opportunistic pathogens. Candida spp. relies on skin or mucosal breach to cause bloodstream infection, whereas Cryptococcus spp. exploit depressed cell-mediated immunity characteristic of advanced HIV infection. Cryptococcosis alone has caused an estimated 1 million cases and 625,000 deaths per year due to central nervous system (CNS) disease among patients with human immunodeficiency virus worldwide. The treatment for both organisms relies on the administration of rapid fungicidal agents.

Flucytosine (5FC) is an antifungal medication used in the management and treatment of systemic and severe Candida and Cryptococcus infections. Flucytosine is 5-fluorocytosine chemically, a fluorinated pyrimidine which is related to fluorouracil and floxuridine. Its structural formula is:

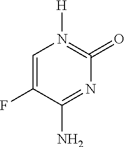

Flucytosine acts as an antimetabolite. It is activated by deamination with the fungal cells to 5-Flurouracil resulting in an inhibition of fungal DNA and protein synthesis. Flucytosine is approved and available in oral dosage form as well as intravenous preparation.

5FC is taken up by fungal cells by cytosine permease and converted into 5-fluorouracil (5FU) by fungal cytosine deaminase. Further metabolism of 5-fluorouracil (5FU) leads to the formation of 5-fluorouridine triphosphate, which is incorporated into fungal RNA, and 5-fluorodeoxyuridine monophosphate, an inhibitor of thymidylate synthetase. This results in inhibition of protein and DNA synthesis in the fungal cell. Side effects of 5FC include nausea, vomiting, diarrhea, bone marrow depression, and hepatotoxicity. The latter two are thought to be due to effects of 5-fluorouracil (5FU) as it is toxic in nature at higher doses. Human cells lack the enzyme cytosine deaminase and are unable to convert 5FC into 5FU. Also, 5FU is toxic and there is always a need to control the conversion of 5FC into 5FU in vivo as well as during the shelf life of formulation.

As oral dosage form, flucytosine (5FC) is available in the form of capsules (ANCOBON®) approved by USFDA. As an alternate dosage form, flucytosine is available as solution for infusion (ANCOTIL) for intravenous or intraperitoneal administration in some countries like Europe and Australia. It is administered in hospital by doctor or nurse at a daily dosage in adults as 200 mg/kg bodyweight divided into four doses over the 24 hours. There is a strict storage temperature requirement for Ancotil to avoid precipitation as well as decomposition during long term storage. It is known that “Ancotil for Infusion should be stored between 18° C. and 25° C. If stored below 18° C., precipitation of Ancotil substance may occur. Prolonged storage above 25° C. could lead to the decomposition of product resulting in the formation of 5-fluorouracil (5FU)”. Thus, stability of flucytosine is affected by temperature.
Further, the cytotoxic and teratogenic degradation product 5-fluorouracil (5FU) may be present in considerable amounts in the product, due to both impurities in the raw material and the formation of 5FU upon sterilisation and inappropriate storage. Since the proposed formulation is an injection product, it is desired that it should be free from visible particulate matters, any precipitates, and impurities. Thus, the manufacturing process of formulation, sterilisation process of formulation and storage of formulation also plays a critical role. Exemplary sterilisation procedures include terminal moist heat sterilization, ethylene oxide, radiation (i.e., gamma and electron beam), and aseptic processing techniques. During the development of formulation of present invention, the inventors subjected the flucytosine solution for infusion in glass containers to the typically recommended method of sterilisation by regulatory authorities i.e., terminal sterilisation method for sealed packages of large volume parenteral (LVPs). However, the inventors found that the flucytosine solution for infusion was unable to withstand the terminal sterilization condition by autoclaving at higher temperatures and resulted in rise of cytotoxic impurity 5FU above acceptable limits.

Therefore, considering the impurity generation of 5-fluorouracil (5FU) and strict storage conditions, development of a sterile, stable, ready-to-use formulation of flucytosine, which is easy to administer to the patient, is challenging. The inventors of present invention have provided a sterile formulation which will overcome the above described shortcomings of capsules and vials of flucytosine, do not have undesirable amount of impurity and which can be administered to a patient by intravenous or intraperitoneal without any intermediate step (s) of dilution/reconstitution or any other type of manipulation (s), which could otherwise compromise the sterility of the solution in the infusion container or could result in an error in dosing to a patient in need thereof

The present invention relates to stable, sterile, ready-to-use aqueous pharmaceutical formulation comprising flucytosine and pharmaceutically acceptable excipients. The formulation is suitable for parenteral administration and is provided in glass container. The invention further relates to the method of manufacture and sterilization of such formulations and using the formulation for treating systemic yeast and fungal infections.

### OBJECT OF THE INVENTION

An object of the present invention is to provide stable, sterile, ready to use, aseptic filled solution for infusion in a glass container comprising flucytosine, its pharmaceutically acceptable salts, and pharmaceutically acceptable excipients.

It is an object of the present invention to provide a sterile ready to use aqueous pharmaceutical formulation comprising flucytosine or its pharmaceutically acceptable salts thereof stored in a glass container, wherein the pharmaceutical formulation comprises about 5 mg/ml to about 20 mg/ml flucytosine or its pharmaceutically acceptable salts thereof in an aqueous sodium chloride solution and wherein the pharmaceutical formulation is manufactured by aseptic fill finish process.

Particularly, the object of present invention is to provide a stable, sterile, ready-to-use, aseptic filled solution for infusion comprising (a) flucytosine (b) buffering agent, (c) isotonicity adjuster, (d) pH adjusting agent and (e) aqueous vehicle stored in presterilized glass container.

An object is also to provide a method of manufacture and sterilisation process of formulation of present invention comprising flucytosine, its pharmaceutically acceptable salts, and pharmaceutically acceptable excipients.

Particularly, another object of the present invention is to provide solution for infusion comprising flucytosine and their pharmaceutically acceptable salts and pharmaceutically acceptable excipients manufactured by aseptic fill finish and filled in suitable presterilized glass container.

Another object of the present invention is to provide formulation of flucytosine which is stable for long shelf life. One of the object of present invention is to aseptic filled solution for infusion comprising flucytosine, its pharmaceutically acceptable salts, and pharmaceutically acceptable excipients in presterilized glass container, which is easy to administer, easy to store and easy to transport.

### SUMMARY OF THE INVENTION

The present invention relates to stable, sterile, ready-to-use aqueous pharmaceutical formulation comprising flucytosine or its pharmaceutically acceptable salts, and pharmaceutically acceptable excipients. The formulation is suitable for parenteral administration and may be provided in a glass container. The invention further relates to the method of manufacture and sterilization of such formulations and using the formulation for treating systemic yeast and fungal infections. In an aspect of present invention, the formulation of present invention is manufactured by aseptic fill finish and filled in presterilized glass container. The formulation of flucytosine of the present invention is stable for long shelf life and is easy to administer, easy to store and easy to transport.

The present invention also relates to a sterile ready to use aqueous pharmaceutical formulation comprising flucytosine or its pharmaceutically acceptable salts thereof that may be stored in a glass container, wherein the pharmaceutical formulation comprises about 5 mg/ml to about 20 mg/ml flucytosine or its pharmaceutically acceptable salts thereof in an aqueous sodium chloride solution and wherein the pharmaceutical formulation is manufactured by aseptic fill finish process. In a preferred embodiment, the formulation comprises about 10 mg/ml of flucytosine or its pharmaceutically acceptable salts thereof.

In one embodiment, the formulation comprising flucytosine or its pharmaceutically acceptable salts thereof further contains about 8.05 mg/ml sodium chloride.

In another embodiment, the formulation comprising flucytosine or its pharmaceutically acceptable salts thereof further contains a buffering agent. In a preferred embodiment, the buffering agent is tromethamine. In a more preferred embodiment, the formulation comprises about 1.5 mg/ml tromethamine.

In one more embodiment, the formulation comprising flucytosine or its pharmaceutically acceptable salts thereof further contains a pH adjusting agent.

In another embodiment, the formulation comprising flucytosine or its pharmaceutically acceptable salts thereof has a pH between about 5.0 to about 8.0. In a more preferred embodiment, the formulation has a pH of about 7.5.

In a further embodiment, the formulation comprising flucytosine or its pharmaceutically acceptable salts thereof is packaged in a glass container. In a more preferred embodiment, the glass container is a glass vial.

In one more embodiment, the formulation comprising flucytosine or its pharmaceutically acceptable salts thereof, when stored for 24 months at 25° C./60% RH contains no more than 0.7% of 5-fluorouracil impurity.

In an embodiment, the formulation comprising flucytosine or its pharmaceutically acceptable salts thereof, when stored for 24 months at 25° C./60% RH contains no more than 0.2% of ethenoflucytosine impurity.

A further embodiment relates to a sterile ready to use aqueous pharmaceutical formulation comprising flucytosine or its pharmaceutically acceptable salts thereof stored in a glass container, wherein the pharmaceutical formulation comprises about 5 mg/ml to about 20 mg/ml flucytosine or its pharmaceutically acceptable salts thereof in an aqueous sodium chloride solution containing about 8.05 mg/ml sodium chloride, about 1.5 mg/ml tromethamine and optionally a pH adjusting agent, wherein the pharmaceutical formulation is manufactured by aseptic fill finish process.

### DETAILED DESCRIPTION OF THE INVENTION

The present invention provides sterile, stable pharmaceutical formulations comprising flucytosine or its pharmaceutically acceptable salts, isomers, racemates, enantiomers, hydrates, solvates, metabolites, polymorphs, and mixtures thereof in a glass container. In an embodiment of the present invention, there is provided a stable, ready-to-use, aseptically filled aqueous solution formulation of flucytosine ready for direct administration to the patient in need thereof without further dilutions.

Flucytosine is chemically designated as 5-fluorocytosine, a fluorinated pyrimidine having a molecular formula C4H4FN3O and a molecular weight of 129.05, and having a chemical structure as given below:

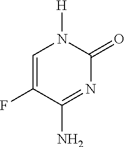

The term “composition” or “formulation” or “dosage form” has been employed interchangeably for the purpose of the present invention and means that it is a pharmaceutical composition which is suitable for administration to a patient or subject. The subject can be an animal, preferably a mammal, more preferably a human.

The term “stable formulations” refers that flucytosine formulations of present invention are physically as well as chemically stable as demonstrated by compliance to acceptable specification when the formulation is stored at ambient temperature, such as between about 18° C. and about 25° C., for a commercially reasonable period of time, such as at least about 1 day, at least about 1 week, at least about 1 month, at least about 3 months, at least about 6 months, at least about 1 year, or at least about 2 years. Suitably, the solution of flucytosine of present invention remains physically stable, with no precipitation or crystallization or color change upon storage and the value of percentage transmittance of the solution remaining greater than 90%, preferably greater than 95% for the shelf life period of 18-24 months when stored at room temperature. Suitably, the solution of flucytosine remains chemically stable when stored at room temperature (about to 18° C. about 25° C.), wherein various parameters such as the drug content (assay of flucytosine) and content of related substances, i.e. known and unknown impurities remains within specified limits such as those specified according to ICH guidelines, upon storage for prolonged period of time such as for at least 12 months, preferably for 18 months, more preferably 24 months or longer. The formulation of the present invention is substantially free of impurities. For purposes of the present invention, “substantially free of impurities’ shall be understood to include flucytosine containing formulations in which the amount of total impurities is less than about 5% of the sum of peak areas of all degradants, as calculated on a normalized peak area response (“PAR) basis as determined by high performance liquid chromatograph (“HPLC) after a period of about 18 months at a temperature of from about 5° C. to about 25° C. The amount of impurities is further calculated as being based upon the original amount of flucytosine (or salt thereof) being present in the composition or formulation. Preferably, the said stable formulations of flucytosine prevent degradation of flucytosine such that not more than 2%, not more than 1%, not more than 0.4%, not more than 0.2% of flucytosine impurity or impurities are formed over the storage period. In yet another preferred embodiment the value of assay of flucytosine remains within the specified limit of 90-110% by weight of the label claim; the highest unknown impurity remains within the specified limit of not more than 0.2%; the known impurity 5-FU remains within the specified limit of not more than 0.50% The total impurities remain below 2.0%, preferably below 1.0% over shelf life.

In some preferred aspects of the invention, the time for which long term storage is contemplated include periods of at least about 18 months or longer with such that the formulation is substantially free of impurities when stored at room temperature.

The term “ready-to-use” refers that formulations of the present invention are premixed compositions that are suitable for administration to a patient without dilution or without any manipulation upon removing the compositions from a sealed packaging container or vessel.

The term “sterile’ formulation, as used in the context of this application, means a formulation that has been brought to a State of Sterility and has not been subsequently exposed to microbiological contamination, i.e., the container holding the Sterile composition has not been compromised. Sterile compositions are generally prepared by pharmaceutical manufacturers in accordance with current Good Manufacturing Practice (“cGMP) regulations of the U.S. Food and Drug Administration.

The term “aseptic fill finish”, as used herein means a process of aseptic sterilization followed by filling under aseptic conditions into a pre-sterilized container. Aseptic sterilization preferably includes passing the formulation through a sterile filter for e.g., 0.22 μm to yield a sterile formulation. Filling of the sterile formulation into pre-sterilized containers under aseptic conditions completes the aseptic fill finish process.

The inventors of the present invention have rigorously conducted experiments with the sterilization process to maintain and control the levels of the impurities, especially the 5-fluorouracil (5-FU) and the ethenoflucytosine impurity within limits.

5-fluorouracil (5-FU) is a cytotoxic compound and is not desired in high amounts. The structure of 5-fluorouracil is as given below:

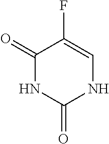

5-fluorouracil (5-FU) is a pyrimidine analogue that competitively inhibits the enzyme thymidylate synthase (TS), thereby creating a thymine deficiency and resulting in inhibition of deoxyribonucleic acid (DNA) synthesis and cytotoxicity. It also inhibits, to a lesser extent, the formation of ribonucleic acid (RNA). These effects are most marked in rapidly growing cells and may lead to cell death. It has been observed that flucytosine is closely related to 5-fluorouracil, and there is a possibility of 5-fluorouracil getting generated as an impurity over the course of time or during manufacturing of flucytosine formulation.
Owing to the cytotoxic nature of the 5-fluorouracil (5-FU) compound, it is of utmost importance to monitor levels of 5-fluorouracil (5-FU) over time, in a formulation containing flucytosine. The inventors of the present invention have rigorously evaluated different sterilization conditions and its effect on level of 5-fluorouracil (5-FU) overtime.

Another impurity of particular importance is the ethenoflucytosine impurity. The chemical name of the ethenoflucytosine impurity is 8-fluoroimidazo[1,2-c]pyrimidin-5 (6H)-one and has the below structure:

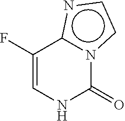

The inventors of the present invention believe to have identified this ethenoflucytosine impurity and through experimentation have been successful to maintain and control the level of this impurity over time. The inventors believe that this impurity has not been reported previously for any flucytosine formulation, let alone, any means for controlling and maintaining the levels.

The inventors of the present invention have evaluated different sterilization methods and conditions and have found that the compositions of the present invention when sterilized by aseptic fill finish method demonstrate lower levels of 5-fluorouracil (5-FU) and the ethenoflucytosine impurity. Aseptic filtration is a sterilization technique that removes microorganisms and other contaminants from the product using filters. This method eliminates the need for exposure to high temperatures and helps to maintain the stability of the final product. Aseptic fill finish is a crucial step in the manufacturing process of pharmaceutical products, as it helps to ensure the quality and efficacy of the final product. By performing this step, the flucytosine formulation can be manufactured in a way that is stable and has a prolonged shelf life. This is especially important in the case of flucytosine, as it is used for the treatment of serious infections and its effectiveness and safety are of utmost importance. The implementation of aseptic fill finish significantly reduces the likelihood of thermal degradation and the formation of cytotoxic impurities, thereby ensuring that the final product remains effective and safe for patients. In one embodiment, the formulation comprising flucytosine or its pharmaceutically acceptable salts thereof, when stored for 24 months at 25° C./60% RH contains no more than 0.7% of 5-fluorouracil impurity. In one more embodiment, the formulation comprising flucytosine or its pharmaceutically acceptable salts thereof, when stored for 24 months at 25° C./60% RH contains no more than 0.2% of ethenoflucytosine impurity.

The formulation of present invention comprises flucytosine or its pharmaceutically acceptable salt in an amount of 5 mg/ml to 20 mg/ml, preferably 10 mg/ml of the total formulation.

The stable injectable pharmaceutical formulations of the present invention include one or more pharmaceutically acceptable excipients. According to the present invention, the formulation of present invention comprises a buffering agent to maintain the pH. The buffering agent may comprise at least one buffering agent selected from the group consisting of citrate (sodium or potassium), acetate, phosphate, malate, lactate, glutamate, titrate, benzoate, glycine, Tromethamine (Tris) and mixtures thereof. The most preferably used buffering agent is tromethamine. In certain embodiments the buffering agent may be present in an amount of about 0.01 mg/ml to about 50 mg/ml, more preferably from about 1 mg/ml to about 30 mg/ml, more preferably from about 1 mg/ml to about 10 mg/ml and most preferably from about 1 mg/ml to about 5 mg/ml. In one embodiment, the formulation of the present invention contains about 1.5 mg/ml of tromethamine. The amount of buffering agent used differs based on the buffering agent used in the formulation.

The parenteral administration of solutions requires that they be adjusted to isotonicity. The isotonicity of the formulation may be obtained by adding an astutely calculated quantity of tonicity agents. As per an embodiment, suitable tonicity adjusting agent which may be used in the formulation of present invention include, but not limited to, sodium chloride, potassium chloride, calcium chloride, sodium hydroxide, potassium hydroxide, dextrose, sodium carbonate, meglumine, sodium lactate, Ringer's solution and lactated Ringer's solution. The preferred tonicity adjusting agent present in formulation of present invention is sodium chloride. The tonicity agent is present in the amount from about 1.0 mg/ml to about 20 mg/ml, preferably from about 5.0 mg/ml to 10 mg/ml, most preferably around 8.05 mg/ml.

According to a particular embodiment, the formulations comprise at least one pH adjusting agent such as sodium hydroxide or hydrochloric acid to adjust the pH to around 6.0 to 8.0. In the case where the buffering agent is constituted of acetic acid/sodium acetate, using additional acetic acid as a pH adjuster is advantageous.

The pH of the formulation changes according to the amount of buffer used. It is preferred to achieve a pH level of between 5.0 and 8.0, preferable to have a pH of about 6.0 to about 7.5, and most preferable to develop a formulation with a pH of about 7.5.

The formulation according to the invention, preferably an infusion solution, is aqueous. Since it is intended for parenteral administration, it preferably contains water, saline, dextrose solution or mixture thereof. Preferably, water for injection is the liquid constituent of the formulation according to the invention.

The present invention relates to stable, sterile, ready-to-use aqueous pharmaceutical formulation comprising flucytosine or its pharmaceutically acceptable salts and pharmaceutically acceptable excipients. The formulation is suitable for parenteral administration and may be provided in a glass container. The invention further relates to the method of manufacture and sterilization of such formulations and using the formulation for treating systemic yeast and fungal infections. In an aspect of present invention, the formulation of present invention is manufactured by aseptic fill finish and filled in presterilized glass container. The formulation of flucytosine of the present invention is stable for long shelf life and is easy to administer, easy to store and easy to transport.

The present invention also relates to a sterile ready to use aqueous pharmaceutical formulation comprising flucytosine or its pharmaceutically acceptable salts thereof that may be stored in a glass container, wherein the pharmaceutical formulation comprises about 5 mg/ml to about 20 mg/ml flucytosine or its pharmaceutically acceptable salts thereof in an aqueous sodium chloride solution and wherein the pharmaceutical formulation is manufactured by aseptic fill finish process. In a preferred embodiment, the formulation comprises about 10 mg/ml of flucytosine or its pharmaceutically acceptable salts thereof.

In one embodiment, the formulation comprising flucytosine or its pharmaceutically acceptable salts thereof further contains about 8.05 mg/ml sodium chloride.

In another embodiment, the formulation comprising flucytosine or its pharmaceutically acceptable salts thereof further contains a buffering agent. In a preferred embodiment, the buffering agent is tromethamine. In a more preferred embodiment, the formulation comprises about 1.5 mg/ml tromethamine.

In one more embodiment, the formulation comprising flucytosine or its pharmaceutically acceptable salts thereof further contains a pH adjusting agent.

In another embodiment, the formulation comprising flucytosine or its pharmaceutically acceptable salts thereof has a pH between about 5.0 to about 8.0. In a more preferred embodiment, the formulation has a pH of about 7.5.

In a further embodiment, the formulation comprising flucytosine or its pharmaceutically acceptable salts thereof is packaged in a glass container. In a more preferred embodiment, the glass container is a glass vial.

In one more embodiment, the formulation comprising flucytosine or its pharmaceutically acceptable salts thereof, when stored for 24 months at 25° C./60% RH contains no more than 0.7% of 5-fluorouracil impurity.

In an embodiment, the formulation comprising flucytosine or its pharmaceutically acceptable salts thereof, when stored for 24 months at 25° C./60% RH contains no more than 0.2% of ethenoflucytosine impurity.

A further embodiment relates to a sterile ready to use aqueous pharmaceutical formulation comprising flucytosine or its pharmaceutically acceptable salts thereof stored in a glass container, wherein the pharmaceutical formulation comprises about 5 mg/ml to about 20 mg/ml flucytosine or its pharmaceutically acceptable salts thereof in an aqueous sodium chloride solution containing about 8.05 mg/ml sodium chloride, about 1.5 mg/ml tromethamine and optionally a pH adjusting agent, wherein the pharmaceutical formulation is manufactured by aseptic fill finish process.

In one embodiment, the present invention provides a sterile, stable, ready-to-use, aqueous pharmaceutical formulation comprising:
(a) 5 to 20 mg/ml flucytosine or its pharmaceutically acceptable salt
(b) a buffering agent to maintain a solution pH between 6.0 and 8.0,
(c) isotonicity adjuster,
(d) pH adjusting agent and
(e) aqueous vehicle
wherein the formulation is manufactured by aseptic fill finish and filled in a presterilized glass container.

The formulation according to the present invention, preferably an infusion solution, can be prepared by conventional processes known to the person skilled in the art.

In one embodiment, the formulation according to the present invention can be prepared by a process, comprising
A) dissolving a tonicity agent and buffering agent in water for injection under stirring;
B) dissolving flucytosine in solution of step (A); and
C) adjusting the pH of the solution, if required and
D) filtering the solution of step (C) through a 0.2 um membrane sterile filter and,
E) filling into glass containers under aseptic conditions.

The ready-to-use pharmaceutical formulations of the present invention are dispensed in a pharmaceutically acceptable container such as glass containers. In one embodiment, the glass container can be an internally coated glass container. In certain other embodiments, the glass container is internally coated with silicon dioxide.

Procedures for filling formulations of the present invention in containers and their subsequent processing is done with care to destroy or eliminate any microorganisms that may be present in the formulations. According to an embodiment, sterile pharmaceutical formulation according to the present invention may be prepared using aseptic processing techniques. All the ingredients used for preparing the formulation of present invention are sterile. Sterility is maintained by using sterile materials and a controlled working environment. All containers and apparatus are sterilized, preferably by heat sterilization, prior to filling. The glass container is then filled under aseptic conditions. Utmost care is taken to avoid the microbiological contamination.

Aseptic sterilization is preferably carried out by passing the formulation through at least one sterile filter, for e.g., 0.22 μm. Several sterilizing grade membrane filters are available for aseptic filtration of water based dosage forms such as cellulose acetate, nylon, polyether sulfone (PES), polypropylene (PP), polyvinyl difluoride (PVDF) and the like.

In a preferred embodiment, the formulation according to the invention is an infusion solution which is prepared for intravenous infusion over a period of time of 2 minutes to 24 hours, more preferably over a period of time of 3 minutes to 6 hours, even more preferably 5 minutes to 1 hour, most preferably 10 minutes to 45 minutes and in particular 20 minutes to 40 minutes.

A further aspect of the invention relates to the formulation comprising flucytosine as described above, preferably an infusion solution, for the treatment of systemic yeast and fungal infections due to sensitive organisms: such infections include cryptococcosis, candidiasis, chromomycosis and infections due to Torulopsis glabrata and Hansenula or the use of flucytosine for the production of a formulation described above for the treatment of systemic yeast and fungal infections due to sensitive organisms: such infections include cryptococcosis, candidiasis, chromomycosis and infections due to Torulopsis glabrata and Hansenula. Preferably the infection is cryptococcal meningitis and severe systemic candidiasis.

In an embodiment, the formulation of present invention is administered in combination with amphotericin-B or fluconazole as determined by doctor considering the patient's condition.

The dose of pharmaceutical formulation of present invention to be administered to the patient in need thereof depends on the age, sex, body weight and condition of patient and can be determined by doctor. In an embodiment, the formulation of present invention may be directly into a vein, through a central venous catheter, or by intraperitoneal infusion. The daily dosage in adults may be 200 mg/kg bodyweight divided into 3-10 doses over the 24 hours, preferably 4-7 doses over the 24 hours. In patients harbouring extremely sensitive organisms, a total daily dose can be adjusted by doctor preferably between 100 to 150 mg/kg bodyweight.

The formulation of present invention is a ‘ready-to-use’ parenteral dosage form which do not require dilution before administration, the volume of the aqueous solution may be from about 50 ml to 1000 ml, preferably 100 ml to 300 ml, most preferably 250 ml.

The formulation of present invention exhibits acceptable stability retains a pharmaceutically desirable appearance without any particulate matter, precipitates and crystallization of flucytosine. Further, the formulation provided herein is suitable for parenteral administration.

The following examples are for the purpose of illustration of the invention only and are not intended to limit the scope of the present invention in any manner whatsoever.

Parenteral formulations of flucytosine of present invention were prepared by using the following methodology:

### Example 1: Formulation of Flucytosine Solution for Infusion

TABLE 1

| Ingredients | Quantity (mg/ml) |
|---|---|
| Flucytosine | 10 |
| Sodium chloride | 8.05 |
| Tromethamine | 1.5 |
| Hydrochloric acid | Qs to pH |
| water for injection | Qs to 1 mL |

Procedure: Dissolve sodium chloride and tromethamine in water for injection under stirring to get clear solution. Add flucytosine gradually to the said solution under continuous stirring. Stir for some time (about 2-3 hours) to ensure uniform mixing of flucytosine in aqueous solution. Measure the pH of solution and if required adjust the pH between 6.0 to 8.0 using hydrochloric acid/sodium hydroxide solution. Adjust the total volume using water for injection and stir to ensure uniform mixing. Fill the solution in a glass container using aseptic fill finish.

### Example 2: Comparative Stability Study of Formulation of Flucytosine Subjected to Different Sterilization Procedures

The formulation of flucytosine of the present invention was subjected to terminal sterilization process by autoclaving at various temperatures as well as subjected to aseptic fill finish process and technique. The assay and impurity profile were determined and presented in table 2 below:

TABLE 2

| | | | 5-FU | Ethenoflucytosine |
|---|---|---|---|---|
| Condition | pH | Assay | (%) | (%) |
| Initial | 6.96 | 103 | ND | ND |
| (Aseptic) | | | | |
| 3 M | 7.04 | 101.2 | 0.084 | ND |
| 6 M | 7.09 | 101 | 0.09 | ND |
| 24 M | 7.10 | 101.5 | 0.471 | 0.08 |
| Initial | 7.17 | 99.4 | 0.317 | ND |
| (Autoclave- | | | | |
| 121° C. for 15 | | | | |
| min) | | | | |
| 3 M | 7.20 | 100.8 | 0.442 | ND |
| 6 M | 7.22 | 101.5 | 0.46 | ND |
| 24 M | 7.27 | 101.4 | 0.823 | 0.11 |

The assay and impurity profile were also determined at various pH and presented in table 3 below:

TABLE 3

| | | | Terminal | Terminal | Terminal | Terminal |
|---|---|---|---|---|---|---|
| | | | sterilization | sterilization | sterilization | sterilization |
| Sterilization | | Aseptic | (initial pH | (initial pH | (initial pH | (initial pH |
| technique | Condition | filtration | 7.6) | 6.8) | 5.0) | 7.8) |
| Sterilization | Temperature | — | 100 | 100 | 100 | 115 |
| Condition | (° C.) | | | | | |
| | Time (min) | — | 30 | 30 | 30 | 15 |
| Properties | pH | 7.58 | 7.43 | 6.73 | 4.93 | 7.65 |
| | Assay (%) | 99.3 | 99 | 99.4 | 99.4 | 99.5 |
| | 5-FU | 0.056 | 0.119 | 0.126 | 0.213 | 0.205 |
| | impurity | | | | | |
| | (%) | | | | | |

Observation: The data of table 2 and table 3 above suggested that the exposure of high temperatures of 121° C., 115° C., and 110° C. for varying periods of time ranging from 2 to 30 minutes as well as different pH conditions can cause thermal degradation and cause potential toxicity due to 5-FU impurity, during the terminal sterilization process. Thus, the above data depicts that the formulation of present invention deteriorated when process of terminal sterilization i.e., autoclaving at high temperature for considerable time was performed. However, the formulation was stable when formulation subjected to aseptic fill finish process and technology for same time.
Thus, it can be concluded that the sterile formulation of present invention is not stable when sterilization process at high temperatures like terminal sterilization or any process other than aseptic fill finish is performed. The important aspect of flucytosine formulation is its manufacturing and stability is crucial for ensuring its effectiveness and usability overtime. The

### Example 4: Stability of Formulation of Present Invention

The formulation of example 1 prepared and packaged in glass container by aseptic fill process and technology under aseptic conditions was subjected to standard storage conditions to determine the stability of present invention. The data has been presented in table 4 below:

| | | | | 5-FU | Ethenoflucytosine |
|---|---|---|---|---|---|
| Condition | | pH | Assay | (%) | (%) |
| Initial | | 7.48 | 101.2 | 0.018 | ND |
| 25° C./ | 3 M | 7.43 | 100.7 | 0.10 | 0.006 |
| 60% RH | 6 M | 7.53 | 100.1 | 0.16 | 0.027 |
| | 12 M | 7.51 | 100.2 | 0.27 | 0.037 |
| | 18 M | 7.36 | 99.3 | 0.398 | 0.071 |
| | 24 M | 7.44 | 100.6 | 0.50 | 0.106 |
| 30° C./ | 3 M | 7.44 | 99.4 | 0.15 | 0.013 |
| 75% RH | 6 M | 7.52 | 99.5 | 0.30 | 0.064 |
| | 12 M | 7.56 | 99.7 | 0.51 | 0.091 |
| 40° C./ | 3 M | 7.46 | 98.1 | 0.50 | 0.072 |
| 75% RH | 6 M | 7.55 | 98.4 | 0.97 | 0.304 |

Observation: From above data, it is observed that the change in pH and assay, osmolality & degradation products in all the stability conditions was in acceptable limits. The data also depicts that the formulation of the present invention is able to keep the levels of the 5-fluorouracil (5-FU) to no more than 0.7% and ethenoflucytosine impurities to no more than 0.2% levels.
Thus, the formulation of present invention is stable at long term condition for the studied parameters.

It will be readily apparent to one skilled in the art that varying substitutions and modifications may be made to the invention disclosed herein without departing from the spirit of the invention. Thus, although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

1. A sterile ready to use aqueous pharmaceutical formulation comprising flucytosine, or its a pharmaceutically acceptable salts thereof, stored in a glass container, wherein the pharmaceutical formulation comprises about 5 mg/ml to about 20 mg/ml flucytosine, or its a pharmaceutically acceptable salts thereof, in an aqueous sodium chloride solution, and wherein the pharmaceutical formulation is manufactured by aseptic fill finish process.

2. The formulation of claim 1, wherein the aseptic fill finish process comprises a step of filtering flucytosine in an aqueous sodium chloride solution through at least one sterile filter followed by aseptic filling into a glass container.

3. The formulation of claim 1, wherein the formulation, when stored for 24 months at 25° C./60% RH contains no more than 0.7% of 5-fluorouracil impurity.

4. The formulation of claim 1, wherein the formulation, when stored for 24 months at 25° C./60% RH contains no more than 0.2% of ethenoflucytosine impurity.

5. The formulation of claim 1, wherein the formulation comprises about 10 mg/ml flucytosine, or a pharmaceutically acceptable salt thereof.

6. The formulation of claim 1, wherein the formulation comprises about 8.05 mg/ml sodium chloride.

7. The formulation of claim 1, wherein the formulation further comprises a buffering agent.

8. The formulation of claim 7, wherein the buffering agent is tromethamine.

9. The formulation of claim 8, wherein the formulation comprises about 1.5 mg/ml tromethamine.

10. The formulation of claim 1, wherein the formulation further comprises a pH adjusting agent.

11. The formulation of claim 1, wherein the formulation has a pH between about 5.0 to about 8.0.

12. The formulation of claim 11, wherein the formulation has a pH of about 7.5.

13. The formulation of claim 1, wherein the formulation comprises flucytosine.

14. The formulation of claim 1, wherein the glass container is a glass vial.

15. (canceled)

16. A sterile ready to use aqueous pharmaceutical formulation comprising flucytosine, or a pharmaceutically acceptable salts thereof, stored in a glass container, wherein the pharmaceutical formulation comprises about 5 mg/ml to about 20 mg/ml flucytosine, or a pharmaceutically acceptable salts thereof, in an aqueous sodium chloride solution containing about 8.05 mg/ml sodium chloride, about 1.5 mg/ml tromethamine, and optionally a pH adjusting agent, wherein the pharmaceutical formulation is manufactured by aseptic fill finish process.

17. The formulation of claim 16, wherein the formulation comprises flucytosine.

18. The formulation of claim 16, wherein the formulation, when stored for 24 months at 25° C./60% RH, contains no more than 0.7% of 5-fluorouracil impurity.

19. The formulation of claim 16, wherein the formulation, when stored for 24 months at 25° C./60% RH, contains no more than 0.2% of ethenoflucytosine impurity.

20. A kit comprising:
a pharmaceutically acceptable glass container and
a sterile ready to use aqueous pharmaceutical formulation;
wherein,
the sterile ready to use aqueous pharmaceutical formulation is stored in the pharmaceutically acceptable glass container,
the pharmaceutical formulation comprises about 5 mg/ml to about 20 mg/ml flucytosine, or a pharmaceutically acceptable salt thereof, in an aqueous sodium chloride solution, and
the pharmaceutical formulation is manufactured by aseptic fill finish process.

21. The kit of claim 20, wherein the glass container is internally coated.

22. The kit of claim 20, wherein the glass container is internally coated with silicon dioxide.

23. The kit of claim 20, wherein the glass container is a glass vial.

24. The kit of claim 20, wherein the pharmaceutical formulation comprises about 5 mg/ml to about 20 mg/ml flucytosine, or a pharmaceutically acceptable salt thereof, in an aqueous sodium chloride solution comprising about 8.05 mg/ml sodium chloride, about 1.5 mg/ml tromethamine, and optionally a pH adjusting agent.

25. The kit of claim 20, wherein the formulation comprises flucytosine.

26. The kit of claim 20, wherein the formulation has a volume of 100 ml to 300 ml and does not require dilution before administration.
